(19) United States
(12) Patent Application Publication
(10) Pub. No.: US 2021/0113765 A1
(43) Pub. Date: Apr. 22, 2021

(54) SMART DEVICE AND SYSTEM FOR TREATMENT OF GASTRIC REFLUX

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventor: Abdullah Muhayl Alotaibi, Riyadh (SA)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 16/655,736

(22) Filed: Oct. 17, 2019

(51) Int. Cl. A61M 5/172; A61B 10/04; A61F 2/04; A61B 5/145; A61B 5/00

(52) U.S. Cl. CPC A61M 5/1723; A61M 2210/105; A61F 2/04; A61B 5/14539; A61B 5/0084; A61B 5/4211; A61B 5/4233; A61B 5/4842; A61M 2205/3306; A61M 2205/3324; A61M 2205/3523; A61F 2002/044; A61B 2010/045; A61M 2205/0244; A61B 10/04

(57) ABSTRACT

A smart device that monitors the condition of the lower esophagus by means of cameras, pH sensors, and other biometric sensors. The device may be equipped with movable arms that facilitate the collection of tissue biopsies from the esophagus as well as a reservoir that contains a medicament such as an antacid or other medicament useful for treating GERD. Data from the cameras and sensors may be transmitted and processed outside of the body and used to diagnose and treat esophageal disorders and can provide early detection of esophageal cancer or to treat GERD.

### BACKGROUND OF THE INVENTION

### Field of the Invention

The invention pertains to the field of gastroenterology and to devices for esophageal monitoring and drug treatment of disorders such gastroesophageal reflux disorder (GERD).

### Description of Related Art

Gastrointestinal reflux disease or GERD is a chronic digestive disease where the liquid content of the stomach refluxes into the esophagus. When the lower esophageal sphincter (“LES”), a ring of smooth muscle that separates the esophagus from the stomach, relaxes abnormally or weakens, acid and enzymes from the stomach can flow into the esophagus damaging the tissues of the esophagus and causing a burning sensation. The LES acts as a natural valve to the stomach, remaining closed until the action of swallowing forces the valve open to allow food to pass from the esophagus to the stomach. Normally the valve closes immediately after swallowing to prevent stomach contents from surging upward. When the LES fails to provide that closure, stomach acids reflux back into the esophagus, causing heartburn.

Common symptoms of acid reflux and GERD are a burning sensation in the chest, difficulty swallowing, and sensation of a lump in the throat. Conventional treatment usually begins with over-the-counter medication to neutralize or control the stomach acid. If the pain is prolonged for a few weeks, a doctor may prescribe other treatments which may include medication and/or surgery. Each year there are more than 3 million cases of GERD diagnosed in the United States and the combined costs of direct and indirect treatment exceed $100 billion a year.

GERD is often diagnosed based on results from a physical examination and from the patient's history. Diagnosis of GERD and its complications is often confirmed by an upper endoscopy where a doctor inserts a thin, flexible tube equipped with a light and camera (endoscope) down a patient's throat to examine the esophagus and stomach. While test results can appear normal even when reflux is present, an endoscopy may detect inflammation of the esophagus or other complications. During some endoscopies a sample of tissue or biopsy of tissue is collected for further testing, for example, a biopsy is obtained for complications such as Barrett's esophagus where esophageal tissue is replaced by tissue similar to the intestinal lining. Other GERD complications include ulcers and strictures of the esophagus, cough and asthma, throat and laryngeal inflammation, inflammation and infection of the lungs, and collection of fluid in the sinuses and middle ear. Patients with GERD often suffer from symptoms which reduce the quality of life including dyspepsia, sore throat, chronic dry cough, hoarse voice, difficulty breathing, nausea and regurgitation, excessive salivation, halitosis, chest pain which may often be confused with a heart attack, stress, insomnia, and avoidance of certain foods such as fats, caffeinated drinks, chocolate, peppermint and alcohol.

Various non-drug therapies to alleviate the symptoms of esophagitis include weight loss if the individual is overweight, not wearing tight clothing that constricts the stomach, and not eating for at least three or four hours before lying down. Other non-drug treatments include avoiding foods that tend to open the LES. These foods include caffeinated beverages, such as coffee, tea and sodas; chocolate; fatty foods and peppermint.

When non-drug treatments are not enough, the individual can take an over-the-counter antacid for the occasional case of heartburn, such as MAALOX®. Also available are over-the-counter acid blockers like TAGAMET HB®, PEPCID®, AXID AR®. or ZANTAC 75®. These drugs serve to block the release of acid into an individual's stomach. Additionally, there are other medicines that help empty the stomach of food and reduce the chances of reflux. These medicines are known as pro-kinetic drugs and include cisapride and metoclopramide.

An additional class of medicaments useful in combating esophagitis is known as proton pump inhibitors. These drugs act to block the molecular “pump” that produces acid in the stomach. These drugs include omeprazole and lansoprazole. Proton pump inhibitors are typically used only in severe cases of esophagitis. They are the strongest known medicines available to counter severe symptoms of esophagitis. Typically, a physician will prescribe a combination of a proton pump inhibitor with a pro-kinetic drug, such as for example, the combination of omeprazole and cisapride

For patients whose esophagitis does not fully respond to any prescription treatment, surgery is an option. The general approach for corrective surgery involves creating a new valve or tightening the existing valve. This procedure is known as “fundoplication” and is used to prevent the back flow of stomach acids into the esophagus. Various fundoplication procedures have been developed to correct GERD and are known as Nissen fundoplication, Belsey Mark IV repair, Hill repair and Dor repair. Each surgical procedure has its own unique attributes; however, each requires an invasive surgical procedure, whereby the individual must endure trauma to the thoracic cavity. The patient remains hospitalized after the procedure for about six to ten days.

Various types of surgically implantable medical devices have been proposed to treat GERD or the GI tract. U.S. Pat. No. 5,887,594 describes a piercing device to retract a section of stomach tissue and form an esophageal valve, U.S. Pat. No. 6,073,052 describes a balloon-like device to prevent GERD, U.S. Pat. No. 10,176,412 B2 describes transponders and sensors for an implantable medical device to monitor signals from the circulatory, respiratory or gastrointestinal systems, such as heart sounds, breathing sounds, or bowel sounds, respectively. Signals may be received by WIFI or Bluetooth™ receivers positioned outside of the body. U.S. Patent Publication 2018/0085605 A1 describes an implantable device using ultrasonic backscatter for sensing a physiological condition for example with a pH, temperature, strain, or pressure sensor. U.S. Pat. No. 10,219,748 B2 describes a gastrointestinal sensor contained in an orally administrable capsule having a tissue capture device for attaching to GI tissue and a biometric sensor for monitoring of the GI tract. U.S. Pat. No. 8,370,078 B2 discloses a remote implantable sensor which bilaterally communicates with an external receiver. The sensor can monitor foods and nutritional products consumed by a patient or the effects of medications. A sensor may also contain a therapeutic reservoir from which a medication may be released. Cao, et al., IEEE Transactions on Biomedical Engineering (2012), describes a wireless and implantable capsule impedance-sensing device that detects the occurrence of reflux episodes.

Existing devices and associated therapies are insufficient for a number of reasons including that they are complicated, costly, require repeated and invasive endoscopic monitoring, do not provide for early detection of conditions such as esophageal cancer, do not provide data over a period of time that could help diagnose and treat a persistent condition such as GERD or other disorders that slowly develop. Major surgery is often required for installation of such devices. Moreover, these devices are not designed to provide mechanical as well as drug based therapies. Non-smart prior art devices do not provide for immediate, automated, or customized therapy based on continuous feedback from sensors such as pH sensors and thus can lead to over or under medication of a patient, in delayed therapy or in patient noncompliance with a therapy regimen.

To address these problems, the inventor designed and developed an easily implantable smart device for diagnosing, monitoring, and treating diseases such as GERD, reflux esophagitis and related diseases such as dyspepsia, esophagitis, esophageal tumor, gastrointestinal disorders, chronic pharyngitis, Barrett's esophagus, esophageal adenocarcinoma, GERD-related pulmonary dysfunctions or other esophageal disorders such as cancer or varices, dysphagia, odynophagia, or other motility disorders, or esophageal strictures or other malformations.

### BRIEF SUMMARY OF THE INVENTION

The foregoing paragraphs have been provided by way of general introduction and are not intended to limit the scope of the following claims. The described embodiments, together with further advantages will be best understood by reference to the following detailed description taken in conjunction with the accompanying drawings.

The invention is directed to a device that monitors the condition of the lower esophagus using cameras and/or pH sensors, and/or other biosensors. The device may also contain movable arms which can help temporarily or permanently anchor the device to the lower esophagus or obtain biopsies of esophageal tissue which the camera components may image. Data from the cameras and sensors may be transmitted and processed outside of the body and used to diagnose and treat esophageal disorders, for example, to provide early detection of esophageal cancer based on imaged biopsies or to treat GERD by administering a medicament.

In some typical embodiments, the movable arms function to take biopsies and optionally have a light-source at their distal ends for better tissue visualization. The device can be anchored temporarily or permanently to the lower esophagus various fixing methods including suturing the edges of the arms to the lower esophageal lining by upper endoscopy approach, by the use of adhesives or other attachment materials, by molding or forming the arms into the body, or by other attachment or forming methods known or developed in the art.

In some embodiments, the device also includes a reservoir of at least one releasable acid neutralizing compound or at least one medicament which can be released in response to detection of particular conditions.

In other embodiments, the device contains a valve, preferably a one-way valve or a valve that can be opened and closed, that permits passage of foods and liquids from the mouth into the stomach, but which prevents acid reflux from the stomach into the esophagus. Typically, the valve or one-way valve are longitudinally oriented in a hollow core of the substantially cylindrical device and run the length of the device.

The device disclosed herein may be operated as a component of smart system that records data output by the device (such as esophageal pH and images), can release acid-neutralizing fluid or medicaments to compensate for stomach reflux in the esophagus, and take images and videos of the lower esophageal tissue. It may be linked via wireless communication to a processor running software that interprets the transmitted images and videos and evaluates pH and sensor readings to produce a log or medical report of the status of the LES and lower esophagus.

The invention also pertains to methods for diagnosing, monitoring, and treating esophageal disorders such as GERD. Use of the system as disclosed herein enhances the standard of care of patients with GERD and other esophageal disorders by avoiding the expense of repeated and costly upper endoscopies, providing early detection and characterization of esophageal disorders including GERD and cancer thus reducing the need for surgery and its attendant risks, providing a more rational dosing and scheduling of medicaments to the patient based on real time or closer to real time assessment of esophageal status, thus reducing overmedication, and improving physician productivity by providing an easily implantable device a system that automates the continual acquisition, recording, and reporting of data describing the condition of the lower esophagus.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1 shows the lower part of the esophagus (10) in relation to the stomach, the position of the sphincter (20), and the stomach (30).

FIG. 2 shows a cross-section depicting the device (100), four cameras (200), four arms (300) and a section of a valve or one-way valve (400).

FIG. 3 shows a cross-sectional view of the device depicting the device (100), cameras (200), arms (300) and a cross section of a valve or one-way valve (400).

FIG. 4 describes some basic functions of the device: obtaining and transmitting images or videos, administration of neutralizing fluids or medications, measurement of pH, and control and controlling biopsies procedures.

FIG. 5 shows a block diagram of certain functions of the internal device and functions of processor elements which are typically external to the device.

FIG. 6 describes a system for receiving camera and biosensor inputs, processing and transmitting the inputs and controlling outputs such as closing a valve or one-way valve or dispensing a medicament or acid neutralizer.

FIGS. 7A-7C describe valves having various shapes. Ball and cage (FIG. 7A), tilting disk (FIG. 7B), and bileaflet (FIG. 7C).

FIGS. 7D-7F depict bileaflet (FIG. 7D) valve and a bileaflet valve with hinges, leaflets and suture ring (FIG. 7E) resembling a cardiac bicuspid valve (FIG. 7F).

FIGS. 7G-7I show trileaflet type valves resembling cardiac tricuspid valve.

FIG. 8 depicts one embodiment of the mechanical/biopsy arms of the device.

FIG. 9 depicts an example of mini- or microcameras that can be used in the device.

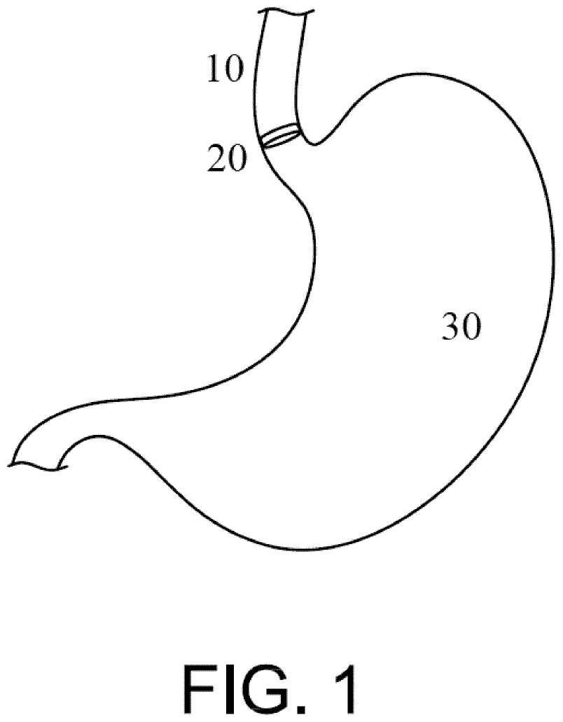

### DETAILED DESCRIPTION OF THE INVENTION

Typically the device of the invention is cylindrical or ring-shaped and sized to snugly fit within the lumen of the esophagus which is about 2 cm in diameter. The device is typically 1, 1.25, 1.5, 1.75, 2, 2.25, 2.5, 2.75 or 3 cm in diameter depending on the anatomy of the patient's esophagus and which can be at least 0.5, 0.75, 1.0, 1.25, 1.5, 1.75 or 2 cm in length. Typically, the device is cylindrical or coin-shaped, but in some alterative embodiments it may be spheroidal or contain one or more inflatable elements to help position it or affix it within the lumen of the esophagus. Typically, the device is sized so that if it detaches and passes into the stomach it can be passed through the small intestine which has a diameter of about 2.5 to 3 cms. The body of the device as disclosed herein may be configured to contain holes or depressions into which arms, cameras or sensors may be seated. It may also include a space for a power supply such as a battery or for positioning data transmission elements or means for transmitting data to a processor outside of the body.

The device is typically 1, 1.25, 1.5, 1.75, 2, 2.25, 2.5, 2.75 or 3 cm in diameter depending on the anatomy of the patient's esophagus and can be at least 0.5, 0.75, 1.0, 1.25, 1.5, 1.75 or 2 cm in length.

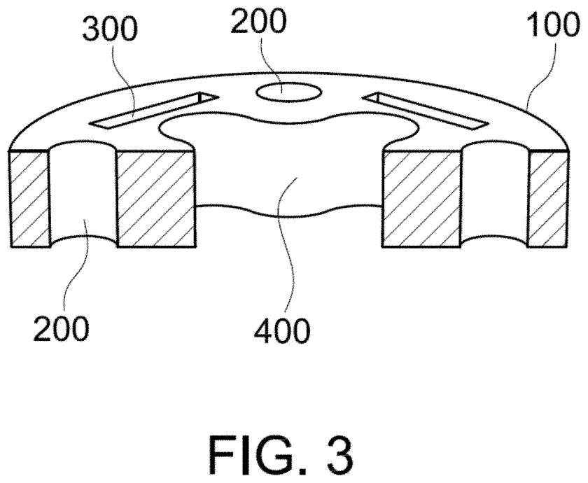

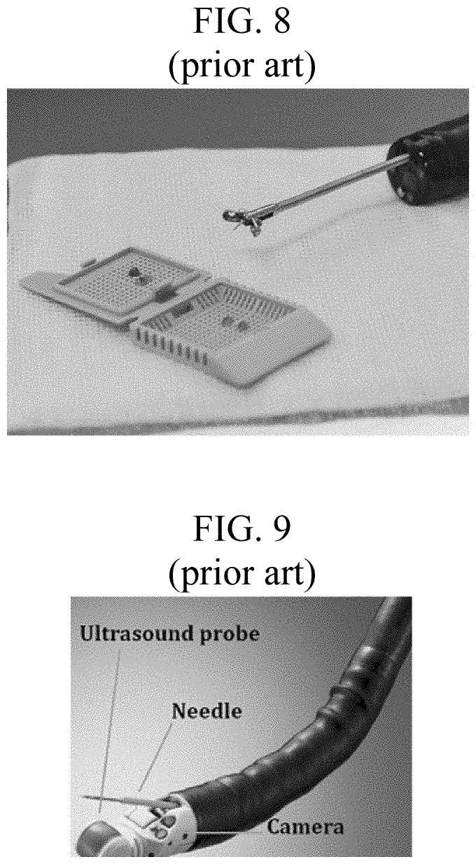

The device is typically affixed to the esophagus by various methods including suturing its edges to the lower esophageal lining by an upper endoscopy approach. The arms are flexible, and will be located within the upper part of the device and under external control, they can be moved to visualize the lower esophageal tissue and take a biopsy if needed. The biopsy arm(s) will be a movable hinged arm; its distal end is the same as or similar to conventional biopsy needles used with upper endoscopy (FIG. 8). The biopsy arm is seated or attached to the device in position 300 shown in FIG. 3. In some embodiments the biopsy arms will house a needle or fine needle for taking the biopsy.

Prior, during or after taking a biopsy sample, the biopsy site and/or sample is photographed for any clear evidence of tissue changes such as redness due to acid reflux. Once a tissue biopsy is taken, it is placed into or stored in a compartment inside of the body of the device. This compartment may contain a magnifying lens to provide further visual detail about the microscopic details of the biopsy sample. In some embodiments, a biopsy sample site can be visualized and photographed by the device and then taken for further external analysis by performing an upper endoscopy.

Preferably the device is fasted to the lower esophagus on or above the gastroesophageal junction above the gastric folds. It may be positioned so as to permit viewing and biopsy of the diaphragmatic hiatus and the squamal columnar junction or the point above the lower esophageal sphincter where the squamous epithelium of the esophagus and the columnar epithelium of the stomach meet.

Preferably, the device comprises components to which microorganisms, saliva, mucosal secretions and food components do not substantially attach or foul, corrode or otherwise degrade and which are not recognized by components of a patient's mucosal or secretory immune system. Such materials include glass, ceramic, metals including titanium, cobalt/chrome and stainless steel, electronic components, and resins. In some embodiments such materials comprise one or more of polyimide, polyethylene, polypropylene, polyvinyl chloride, epoxy, polyurethane, polycarbonate, acrylic, polystyrene, acrylonitrile butadiene styrene (ABS), nylon, delrin, polyethylene terephthalate (PET), polyether block amide, fluorinated ethylene-propylene (FEP) or polytetrafluoroethylene (PTFE) and silicones.

In some embodiments, the device or its components such as body, arms, cameras, reservoirs or sensors may comprise a flexible or semi-flexible material. Body materials may comprise latex, nitrile, plastics, polymers, rubber materials or other materials known or developed for use in medical applications. Likewise, for other endoscopy applications, materials may be plastics, rubbers, polymers or other materials suitable for such applications, such as materials for corrosive environments, contaminated environments, toxic environments, and the like. In some embodiments, the device or its components may comprise a rigid or substantially rigid material rather than a flexible material.

The device can minimize acid reflux by providing physical or mechanical support to the esophagus and by narrowing the anatomical area at the lower esophageal sphincter.

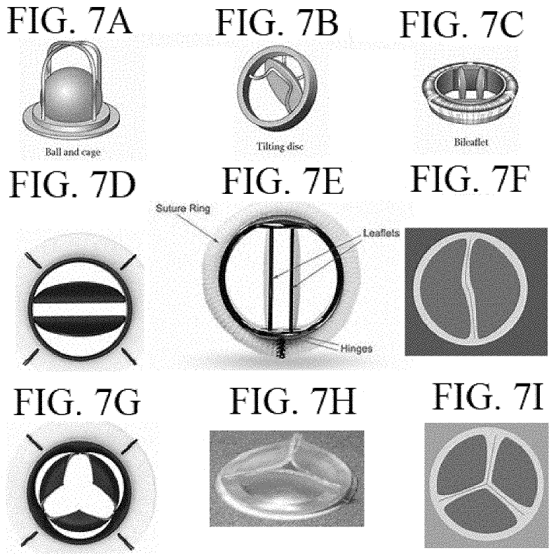

In some embodiments, the device contains a valve, preferably a one-way valve that blocks acid reflux from the stomach when closed. The valve may be opened to permit passage of food into the stomach or closed to block acid reflux for example in response to predetermined conditions in the esophagus. In some embodiments the valve may structurally resemble a heart valve, such as a one-way heart valve or the valves depicted by FIGS. 7A-7I. Such one-way valves, their structures, components and material used to make them are described by and incorporated by reference to Kostrzewa, B; Rybak, Z (2013). “History, present and future of biomaterials used for artificial heart valves”. Polimery W Medycynie. 43 (3): 183-189. PMID 24377185. In some embodiments a valve or one-way valve used in the device disclosed herein will be a mechanical valve. These include caged ball, tilting disc, bileaflet and trileaflet mechanical valves. A mechanical valve may comprise or be made of various materials including metals, ceramics and polymers, e.g.: stainless steel, titanium, silicone, pyrolytic carbon. It may be made of metal or carbon surrounded with a polyester knit fabric-covered ring. The device or valve component of the device as disclosed herein may comprise a suture ring which may be reinforced with a hard flexible plastic or with a metal component, such as with a metal ring or wire. Examples of various types of valves patterned on cardiac valves appear in FIGS. 7A-7I.

The device or valve may operate in conjunction with the LES to provide support or supplement its function in blocking reflux. In the normal anatomy of the upper GI tract, the LES is located at the lower end of the esophagus and prevents the acidic contents of the stomach from returning to the esophagus. This sphincter loosens and becomes less effective in blocking acidic reflux from the stomach in GERD patients. The device as equipped with a valve compensates for this loosening and loss of sphincter function using a valve to block exposure of the lower esophagus to exposure to acid and enzymes from the stomach and maintain or return to its normal state. While one-way valves are used in engineering and have been used in heart surgery, they have not previously been incorporated into an anti-reflux device. A valve or one-way valve is configured so as to permit passage of chewed food or liquids through the esophagus and into the stomach but to block acid reflux. In some embodiments, a one-way valve is configured to always permit passage of food and liquid into the stomach, but to always block acid reflux into the esophagus. In other embodiments, the one-way valve may be turned on or off, for example, by signals from the processor in response to conditions in the esophagus such as an acid pH.

A one-valve may contain at least two cusps on each side of the device (e.g., resembling a bicuspid heart valve) or three evenly spaced cusps (e.g., resembling a tricuspid heart valve), and it will operate continuously or unfold intermittently in response to acid reflux measured by the pH sensors.

For example, in continuously operating a one-way valve, the cusps are flexible and can open to the stomach under the light pressure of the swallowed food to allow its passage to the stomach. Similar to mechanical heart valves, the valve can open in one direction under the influence of a pressure gradient. In another embodiment the valve mechanism can retract inside of the device and then re-fold.

In the case of intermittently opening one-way valves, the pH sensor(s) after detection of acid reflux to the lower esophagus, signal the valves to close and prevent further acid reflex for a specified period.

The device will be flexible in design, able to expand and shorten and can be made from an easily extending material. The device will be able to expand along with esophageal wall expansion due to swallowed food and it will shrink to its original size after the passage of the food to the stomach.

The device as disclosed herein in addition to containing pH and other biosensors may include a drug delivery system that can deliver drugs or meter drug dosages in response to pH or other biosensor readings. One example of such a system is a micro-electromechanical system (MEMS)-based drug delivery device. The use of techniques created for the electronics industry has enabled the production of the micro-reservoirs, micropumps, valves and sensors needed to make miniaturized devices; Staples, M., Daniel, K., Cima, M. J. et al. Pharm Res (2006) 23: 847. https://doi.org/10.1007/s11095-006-9906-4; Staples et al., 2006, Ngoepe et al., Sensors 2013, 13(6), 7680-7713; https://doi.org/10.3390/s130607680 (both incorporated by reference). These devices may be designed to sense, monitor, mix, pump and control the flow of small amounts of medicaments; see Nisar, et al., Sensors and Actuators B: Chemical 130(2), 28 Mar. 2008, Pages 917-942 (incorporated by reference). The inclusion of a drug delivery system in the device reduces the risk to the patient and the liability to the hospital of overdosing/underdosing a patient while allowing the patient to receive the right drug dosage at the right time as determined by sensor and image data acquired by the device as disclosed herein.

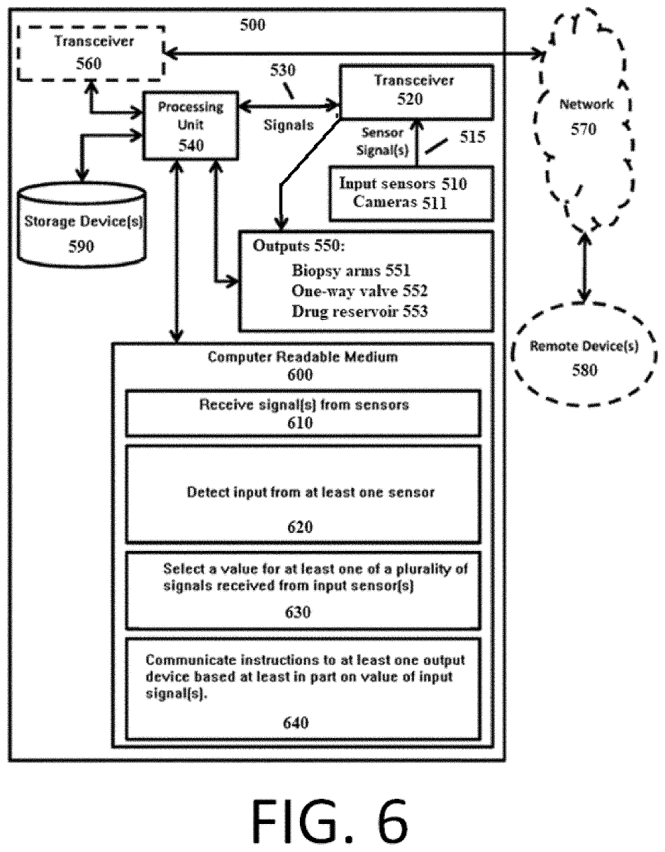

One embodiment of the invention is directed to a device configured for positioning, anchoring, or implanting in the lower esophagus comprising at least one sensor for measuring pH, a transceiver, a power source, and a reservoir for at least one medicament, wherein the sensor and reservoir are operably connected to the transceiver. In one embodiment, the sensors and transceiver are configured as shown by FIG. 6.

The device as disclosed herein is positioned or configured to fit in the lower esophagus and above the stomach. Typically, the device has a substantially cylindrical and hollow axial cross-section which permits it to snuggly fit within the cylindrical esophagus. In some embodiments, the device has a diameter of about 1.5 to 2 cms, a length of about 2 to 3 cms, and a longitudinal hollow core about 1 to 1.75 cms in diameter.

The device may be positioned or affixed to tissue of the lower esophagus where it may be aligned with or positioned immediately above or below the lower esophageal sphincter. The device may be disposed in the body by attachment to the esophagus, such as by use of adhesives or other attachment materials, by molding or forming into the body, or by other attachment or forming methods known or developed in the art. The device is typically positioned on the lower esophageal sphincter for a patient having normal esophageal anatomy.

The device may be positioned, anchored or attached inside of the lower esophagus through the mouth. Typically it is positioned and attached to the inner lining of the lower esophagus. In some embodiments, the arms of the device may be used to affix it within the lumen of the esophagus. The attachment is typically performed endoscopically and prior to the procedure the patient may be given a local anesthetic or IV sedation.

Once attached, the device can send a wireless signal to a receiver outside of the body which can be linked to a data processor to which photographic or sensor data is directed. It is not necessary to remove or recover the device in order to obtain the sensor data from the device. The device may also receive wireless signals from outside the body, for example, a signal to open or close the valve or one-way valve or to release an acid neutralizer or medicament.

The device can be attached temporarily or permanently to the lower esophagus by several fixing methods including suturing its edges to the lower esophageal lining by an upper endoscopy approach, use of adhesives or other attachment materials, by molding or forming into the body, or by other attachment or forming methods known or developed in the art. The device is positioned inside of the esophagus and its arms are retractable and can fold into the body of the device then extend for lower esophageal tissue examination and biopsy taking. A light-source might be attached to the distal part of the arms as well as a biopsy needle.

Once positioned, anchored or implanted, the device may be left in position long enough for initial readings to be taken, for particular clinical procedure to be taken, or for a longer period of time. It may be left in place for 1, 2, 3, 4, 5, 6, 7 or more days. When left in place for a period of days, the drug reservoir in the device may be configured with a port through which it can be endoscopically refilled and the device may be endoscopically cleaned for example, by spraying it with water, saline, or another physiologically acceptable solution. It may also be configured to permit endoscopic replacement of the power source or to permit endoscopic cleaning of the cameras, sensors, reservoirs, arms and body of the device.

Depending upon the utilized material and the medical response, the device might be applied for a short duration (months) or long period (years). The device can be cleaned and refilled by several methods including by a procedure conducted by upper endoscopy.

In some embodiments, other types of biosensors may be substituted for or included with the pH sensor. These include biometric sensors such pressure, impedance, or temperature or sensors for physiological markers of gastroesophageal or duodenogastric reflux, such as bile, sodium, pepsin or pepsinogen (an inactive form of pepsin). In some cases sensors for pancreatic enzymes such as trypsinogen, trypsin, chymotrypsinogen, chymotrypsin, elastase, carboxypeptidase, pancreatic lipases, bile acids like deoxycholic acid, nucleases and amylases may be included. Other GERD biomarkers are incorporated by reference to Kia, et al., Biomarkers of Reflux Disease, Clin. Gastroenterol. Hepatol. 14(6):790-797 (2016).

These sensors are position so as to measure pH or other analytes or conditions in the esophagus, typically above the position where the device is positioned or implanted, though in some embodiments, sensors may be placed or exposed either above or below the position of the device. The sensors are connected to a transceiver which permits their signals to be transmitted to a processor as shown for example in FIG. 6.

The device typically contains one or more reservoirs that can release medicaments including drugs used to treat esophageal disorders as well as materials that neutralize acid refluxed from the stomach into the esophagus. These reservoirs are operably connected to the transceiver so they can be activated to release a particular dose or type of medicament at a particular time according to user or programmed instructions. The reservoir or reservoirs may contain and release or spray one or more medicaments into the esophagus. Medicaments include acid neutralizing solutions or antacids that can raise the pH in the esophagus above 4, 4.5, 5, 5.5, 6, 6.5, 6.6, 6.7, 6.8, 6.9, 7, 7.1, 7.2, 7.3, 7.4, or 7.5. A medicament may also comprise a foaming agent, H2 blocker, a proton pump inhibitor, or a prokinetic drug. The reservoir may be configured to release a medicament above the device into the esophagus or below the device into the stomach.

Additional equipment, tests, or materials may be incorporated into the invention, for example, to further analyze a biopsy. These include, but are not limited to the following: testing the DNA, chemical or immunological components of the abnormal cells, stains to further exam the cellular part of the tissue such as healthy vs. dysplastic cells, as well types of immune cells infiltrates (lymphocytes, eosinophils, etc.). Additionally, materials and chemicals to exclude or inhibit undesirable microorganism such as bacteria, viruses, or fungi can be instilled.

In some embodiments, the device may administer live microbes to restore the natural microbial ecology of the esophagus, especially the lower or distal esophagus. These include members of the phyla Firmicutes, Bacteroides, Actinobacteria, Proteobacteria, Fusobacteria, and TM7 as well as specific microorganisms known to populate the esophagus including those described by Pei, et al., Proc Natl Acad Sci USA. 2004 Mar. 23; 101(12): 4250-4255 (incorporated by reference). Similarly, the device may contain antibiotics or microbiostatic agents to remove or inactivate harmful microorganisms, such as Candida, Herpesvirus, Cryptococcus, Histoplasma, or H. pylori from the esophagus.

The device as disclosed herein is configured to store these and release them when the pH level or other indictors of GERD, GI damage or abnormal esophageal conditions are detected. These medications may be stored and released in a solid, liquid or gaseous form and a preferably released into the esophagus as a liquid or spray to neutralize acid or into the stomach in combination with a suitable pharmaceutical carrier or excipient. Abnormality can be measured in relation to a sensor or image data from an otherwise identical healthy subject who does not suffer from GERD or another esophageal disorder or condition.

Medicaments include, but are not limited to antacid agents, such as aluminum hydroxide, magnesium hydroxide, calcium carbonate, magnesium carbonate, or aluminum-, and calcium-, magnesium- or sodium-bicarbonates; proton pump inhibitors, such as dexlansoprazole, esomeprazole, pantoprazole, rabeprazole, lansoprazole, or omerprazole; H₂ antagonists, such as nizatidine, cimetidine, ranitidine, or famotidine; prokinetic agents, such as benzamide, cisapride, domperidone, itopride, mosapride, metoclopramide, prucalopride, renzapride, tegaserod, mitemcinal, levosulpiride, cinitapride, or linaclotide. Other medicaments include probiotics such as Lactobacillus or Bifidobacterium, including L. rhamnosis and B. lactis, alginic acid or alginates, or chitosan; flavonoids, anthocyanosides, or plant extracts; antioxidants such as vitamin A, C or E, and anti-inflammatory drugs such as NSAIDS or aspirin, naproxen, diclofenac, celecoxib or sulindac, oxaprozin, piroxicam, indomethacin, meloxicam fenoprofen or diflunisal; and combinations of two or more of these.

In preferred embodiments, the device may comprise a replaceable pack or cartridge containing the medicament or a port through which a reservoir may be reloaded with a medicament, drug or neutralizing compound, for example, via endoscopic reloading. In some embodiments, the device has separate compartments for different medicaments, drugs or neutralizing compounds, for example, it may have one compartment the releases a proton pump inhibitor in response to certain sensor input and a separate compartment that releases an acid neutralizing solution in response to a low esophageal pH level or in response to an above normal accumulation of stomach acid. The compartment holding the medicament, drug or neutralizing compound may be configured to comprise release ports on one or both sides of the device, for example, to deliver a drug such as a proton pump inhibitor into the stomach and an acid neutralizing solution into the esophagus. A drug may be actively released, for example, by ejecting it or spraying it out of a nozzle or port or passively released, for example, by exposing it to the fluids in the esophagus or stomach and permitting it to dissolve.

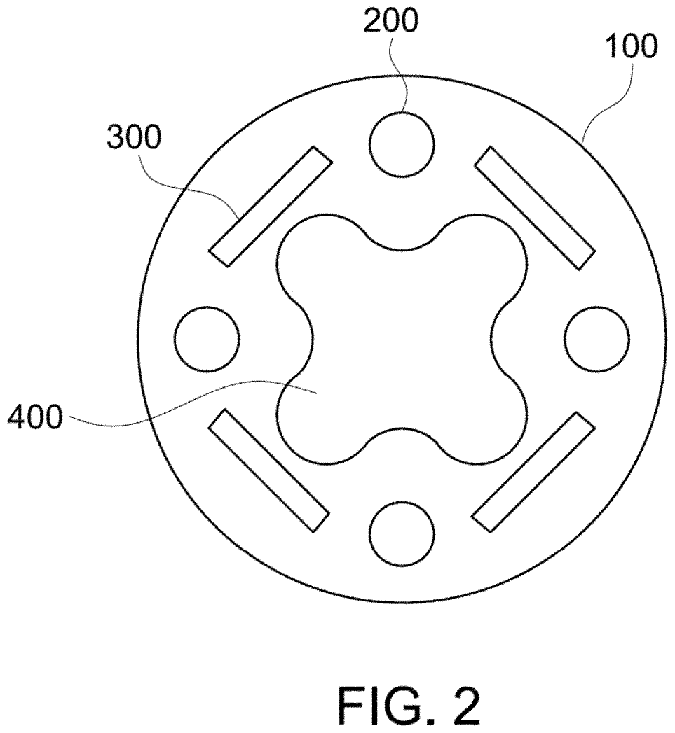

In some embodiments, the device includes 1, 2, 3, 4, 5, 6, or more cameras which are preferably spaced along an outer circumference of the device as shown for example in FIGS. 2 and 3. Any small size camera, such as those conventionally used in a medical device (see FIG. 9) may be incorporated into the device of the invention. One skilled in the art can select a design of shape suitable for use in a particular patient or for visualization of particular esophageal conditions. For cellular examination, the camera may be associated with a magnifier lens that provides at least 5, 10, 20, 30, 40, 50× or more magnification.

Like the sensors, the cameras are also operably connected to the transceiver so that images and data can be transmitted to a processor. A transceiver may amplify, filter or clarify a signal from a camera or other sensor so that it can be received by a processor which is typically located outside of the body, but in some embodiments may form part of the implanted device. When the device includes one or more cameras and biopsy arms, the results of the biopsy may be imaged and transmitted to the processor. These imaging results may be used to determine whether predetermined conditions have been met that would trigger transmission of a signal from the processor to the device to release a medicament or to open or close a valve. For example, imaging data from the camera showing a color change or morphology changes in biopsied tissue associated with inflammation may be used to trigger transmission of signal to release a medicament that ameliorates inflammation, such as an antacid or anti-inflammatory drug such as an NSAID or cortisone. Similarly, such imaging data can trigger transmission of a signal to close a valve or to activate a one-way valve in embodiments having such a feature to prevent retrograde flow of stomach contents into the esophagus. Other preselected conditions may allow the valve to remain open so as to permit normal passage of food and liquid into the stomach. The device's camera(s) monitor and can photograph the lower esophagus and transmits these images to an external processor. The processor can compare the received images to photographs of normal or abnormal esophageal tissue and then transmit a signal to the device, for example, to release an acid neutralizer or medicament when abnormal tissue or inflammation is detected, especially, if detected in combination with data indicating an acidic pH in the lower esophagus from pH sensors. Alternatively, a doctor or medical professional, or in some cases the patient, may review and compare the transmitted images or data for biomarkers indicative of GERD or other disorder and send a signal to the device. In some embodiments, the device may be used for post-treatment surveillance or continued monitoring of a previously treated patient.

In other embodiments, the device includes 1, 2, 3, 4, 5, 6 or more arms which are designed and configured to take biopsies of esophageal tissue. The arms are also operably connected to the transceiver so that they can be activated by a user or a predetermined program when a biopsy is needed. Multiple arms are preferred so that a more representative biopsy sampling of esophageal tissue can be obtained which reduces the risks missing inflamed or abnormal areas of tissue. In one embodiment as shown by FIGS. 2 and 3, the device has 4 movable arms spaced at approximately equal distances around its perimeter. Once obtained a tissue sample may be viewed through a magnifying lens inside the implanted device to provide a preliminary view as to whether the tissue is normal or abnormal. In some embodiments, biopsy samples may also be collected by an upper GI endoscopy or EGD to increase sampling accuracy, decrease EGD duration, and provide earlier detection of transformation of normal tissue into abnormal tissue.

The arm's length is in millimeters and shape might be rectangle or cylindrical. It may consist of two parts (short and long pieces). The small piece is connected proximally to the device and distally to the long piece.

The small piece moves in uni-direction up and down from the upper part of the device.

The long piece is connected proximally to the small piece of the arm, flexible at the junction with the small part as well it able to move in multiple directions. The distal end of the long portion might have a camera, light source, and/or a needle for biopsy. Alternatively, the cameras and the light source can be positioned along the outer circumference of the device.

Another embodiment of the device disclosed herein comprises a valve, preferably a one-way valve, typically in the center of the device as depicted by FIGS. 2 and 3 that is oriented so as to prevent retrograde flow of stomach contents into the esophagus, but permit passage of material from the esophagus into the stomach. The valve is operably connected to the transceiver through which it receives signals from the processor to open or close. In embodiments using a one-way valve, the valve typically continuously functions as a one-way gate that prevents acid reflux from the stomach from passing upward into the stomach, though in some embodiments, the one-way valve may be activated and then become functional after receipt of a signal from another part of a system comprising the device or the one-way valve may be opened or closed by a signal via the transceiver. The one way valve may be activated or opened or closed when conditions detected by the pH, other biosensors or cameras indicate a significant and continuous need to prevent reflux from the stomach, for example, when the LES cannot fully function.

In some embodiments, the device may comprise a sensor that determines whether the lower esophageal sphincter (LES) is open or closed and detect openings and closings of the sphincter, for example, it can detect transient LES relaxations. In other embodiments, the device will contain a sensor that detects whether the upper esophageal sphincter is open, partially open or closed. By monitoring the positions of both the upper and lower esophageal sphincters, the device can be programmed to recognize when a patient is swallowing while eating or drinking to permit normal passage of food and liquid from the mouth through the esophagus into the stomach. Similarly, it can detect abnormal opening and closing of either sphincter, for example, transient opening of the lower esophageal sphincter during reflux. The device may also include sensors which detect esophageal contractions that force stomach acids back into the stomach.

Another embodiment of the invention is directed to a smart system which includes an implanted device as disclosed herein and data receiving, processing, memory, and remote components. The system is configured to receive sensor and camera image/video inputs from an implanted device, transmit the data to a processor which evaluates the data and then communicates instructions to biopsy arms, one-way valve and drug reservoir located on the implanted device. In some embodiments the processor converts signals from the sensors or cameras into a computer readable form and communicates with a network and/or remote devices such as a printer, display, computer, laptop or mobile phone.

In some embodiments, the system includes an alarm that notifies a user of a particular set of conditions detected by the sensors and cameras or notifies the user that certain actions like dispensing a medication are impending, underway, or complete. For example, the alarm may notify a user of the presence of an acidic pH in the esophagus and the impending release of an acid neutralizing medicament by the reservoir. An alarm may function by sensation, an auditory signal such as a ringtone on a wireless phone or beeper, or visual output such as a light or series or array of lights, a display on a screen such as a wireless phone screen, or by tactile signaling. The system may also notify the user of opening or closing of a one-way valve to permit passage of food through the device into the stomach or to secure the esophagus from exposure to reflux of stomach contents. Similarly, when image data indicates inflammation or abnormal or cancerous tissue in a biopsy, the processor can record the abnormality and report the abnormality to medical personnel. The processor can also be programmed to intermittently (or “as needed”), periodically or continuously obtain pH and other sensor readings or obtain images/videos or to conduct biopsies when particular conditions are present; or to intermittently, periodically or continuously release medicaments or open or close a one-way valve.

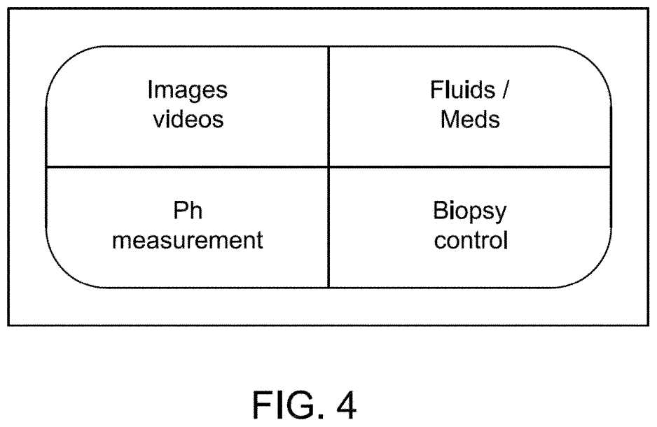

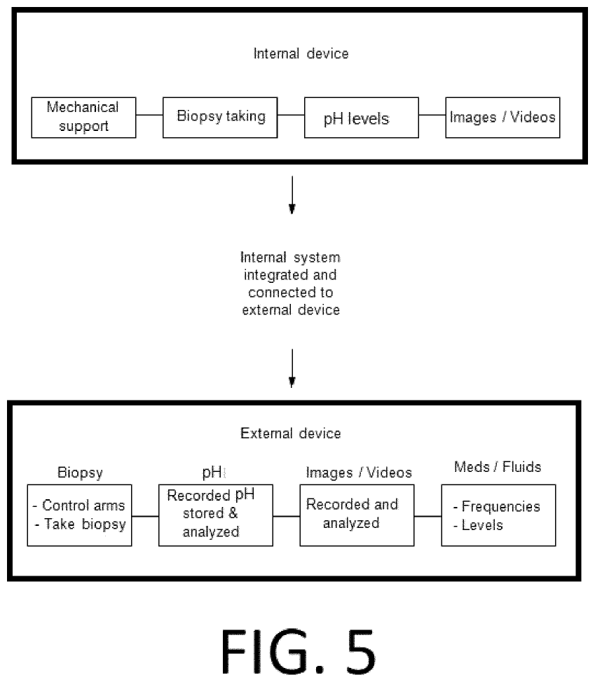

One embodiment of such a system is shown by FIG. 6. As shown, the system receives one or more sensor/image signals 515 from input sensors 510 and cameras 511 on an internal device positioned in the lower esophagus, transmits these signals to a transceiver 520 and then to a processing unit 540, which processes the raw data from the sensors and cameras. Once sensor or image signals are received by processor 540 they may be encoded into a computer readable medium 600 as shown by FIG. 5. The encoded signals or instructions may be transmitted or communicated to devices recognizing them. As described by FIG. 6, in some embodiments, the processor 540 employs a computer readable medium 600 to receive signals from sensors 610, detect input 620 from at least one sensor, such as a pH sensor or camera image, selects a value for at least one of a plurality of signals 630 received from input sensors 510 and cameras 511 and communicates 640 instructions to at least one output 550.

Once sensor or image signals are received by processor 540, the processor transmits instructions via transceiver 520 to outputs 550 (e.g., to biopsy arms 551, one-way valve 552 and/or drug reservoir 553); and/or transmits data via transceiver 560 to an external network 570 and to remote devices 580. Network transceiver 560 may also receive instructions from an external network of remote device and transmit these via processor 540 to outputs 550. The transceiving units of the system 520 and 560 include at least one transmitter and at least one receiver. In some embodiments, a single transceiver is used in place of transceivers 520 and 560 and can receive and send signals to elements of the implanted device and to an external network or remote device.

Typically, the input sensors 510, cameras 511, outputs 550 and transceiver 520 are incorporated in the implanted device and the processing unit 540, network transceiver 560 and storage device(s) 590 are located outside of the body. When processor 540 is located on the implanted device, it may send instructions directly to the output devices 550.

Sensor readings and images may be recorded by the system, for example, stored in memory or a database attached to the sensors, processor or other components of the system that can receive data. According to some embodiments, the at least one storage device 590 may be communicatively coupled to system 500 through employment of a wired and/or wireless network. The at least one storage device 590 may be managed through employment of a cloud service, a web-based electronic data capture system, a web application, a mobile device application, a mobile device operating system, a virtual machine, combinations thereof, and/or the like.

Another embodiment of the invention is directed to a method for diagnosing or monitoring a patient for GERD or another esophageal disorder comprising positioning the device disclosed herein in the lower esophagus of the patient, receiving output from the pH sensor and camera on the device, and optionally processing or storing the output. In some embodiments this method is directed to treating a patient having GERD or another esophageal disorder and includes selecting such a patent when the pH of the esophagus pH is less than 3.5, 4, 4.5, 5, 5.5, 6, 6.5, 7, 7.1, 7.2, 7.3, 7.4 or 7.5, and/or when a camera image indicates inflammation or abnormal tissue in at least one area of the lower esophagus. This method may further include obtaining a biopsy of esophageal tissue using one or more biopsy arms on the device, and selecting a patient having GERD or another esophageal disorder is selected when the pH of the esophagus pH is less than 3.5, 4, 4.5, 5, 5.5, 6, 6.5, 7, 7.1, 7.2, 7.3, 7.4 or 7.5 and/or when a camera image indicates inflammation or abnormality in the lower esophagus, and/or a camera image indicates eosinophilia of the lower esophagus, and/or when a camera image shows precancerous or cancerous cells in a biopsy or other disorders or abnormal conditions of the esophagus such as malformations. This method is preferably applied to patients suspected of having GERD, but may be applied to other disorders or conditions affecting the esophagus. In some embodiments the medicament comprises an acid neutralizing material and/or a foaming agent, H2 blocker, a proton pump inhibitor, or a prokinetic drug.

Another embodiment of the invention is directed to method for treating a patient for GERD or another esophageal disorder comprising positioning the device as disclosed herein in the lower esophagus of the patient, preferably by a minisurgical procedure conducted through the mouth and esophagus, sending output from the pH sensor or image output from a camera on the device to the transceiver, and when a pH of less than 3.5, 4, 4.5, 5, 5.5, 6, 6.5, 7, 7.1, 7.2, 7.3, 7.4 or 7.5 is detected in the esophagus, delivering a signal to the releasable reservoir to release the medicament and/or delivering a signal to a one-way valve on the device to close thus preventing acid reflux from the stomach into the esophagus. This method is conveniently applied to patients suspected of having GERD, but may be applied to other disorders or conditions affecting the esophagus.

Terminology used herein is for the purpose of describing particular embodiments only and is not intended to limit the invention.

The headings such as “Background” and “Summary” and sub-headings used herein generally organize the topics within the present invention and do not limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience and no inference should be drawn that the material must necessarily or solely function in accordance with its classification herein when it is used in any given composition.

Links in the disclosure are disabled by spelling out or deleting “http”, or by insertion of a space or underlined space before or after www. Unless otherwise specified, the content of the link is that which was available via the link on the effective filing date of the application.

As used herein, the singular forms “a”, “an” and “the” include the plural forms as well, unless the context clearly indicates otherwise.

It will be further understood that the terms “comprises” and “comprising,” when used in this specification, specify the presence of stated features, steps, operations, elements, and/or components, but do not preclude the presence or addition of one or more other features, steps, operations, elements, components, and/or groups thereof. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

As used herein, the term “and/or” includes any and all combinations of one or more of the associated listed items and may be abbreviated as “/”. For example, “A and/or B” describes A by itself, B by itself, and both A and B together.

Numbers used in the specification and its examples unless otherwise specified may be read as if prefaced by the word “substantially”, “about” or “approximately,” even if the term does not expressly appear unless otherwise specified.

The phrases “substantially”, “about” or “approximately” may be used when describing a magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc.

Disclosure of values and ranges of values for specific parameters including temperatures, molecular weights, weight percentages, etc. are not exclusive of other values and ranges of values useful herein. Two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

Unless apparent from the context or unless otherwise specified, all compositional percentages are by weight of the total composition.

As used herein, the words “preferred” and “preferably” refer to embodiments of the technology that afford certain benefits under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

Although the terms “first” and “second” may be used herein to describe various features/elements (including steps), these features/elements are not limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention. A first or second or subsequent element may be the same or different from another element.

Spatially relative terms, such as “under”, “below”, “lower”, “over”, “upper”, “in front of” or “behind” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (e.g., rotated clockwise or counterclockwise 30, 45, 60, 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature or not have these features.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A device having a substantially cylindrical and hollow cross-section configured to fit inside the lower esophagus comprising:
at least one sensor for measuring pH,
at least one camera positioned on the device to take photographs of the inside of the lower esophagus,
a transceiver,
a power source, and
a reservoir containing at least one releasable medicament,
wherein the sensor and reservoir containing a releasable medicament are operably connected to the transceiver.

2. The device of claim 1 that has a diameter of about 1.5 to 2 cms, a length of about 2 to 3 cms, and a longitudinal hollow core about 1 to 1.75 cms in diameter

3. The device of claim 1, further comprising:
at least one sensor for pepsin or pepsinogen operably connected to the transceiver.

4. The device of claim 1, further comprising: four cameras spaced at approximately equal distances around an outer circumference of at least one end of the substantially cylindrical device operably connected to the transceiver.

5. The device of claim 1, further comprising:
at least one movable arm configured to take a biopsy of tissue in the lower esophagus, wherein said arm is operably connected to the transceiver.

6. The device of claim 1, further comprising: four movable arms spaced at approximately equal distances around a circumference of the device which are configured to take a biopsy of tissue in the lower esophagus, wherein said arms are operably connected to the transceiver.

7. The device of claim 1, further comprising:
a valve longitudinally oriented inside a hollow core of the substantially cylindrical device so as to permit passage of material from the lower esophagus into a stomach when open, wherein said valve is operably connected to the transceiver.

8. The device of claim 1, further comprising:
a one-way valve longitudinally oriented in a hollow core of the substantially cylindrical device so as to prevent retrograde flow of stomach contents into the lower esophagus, but permit passage of material from the lower esophagus into a stomach.

9. A system comprising the device of claim 1 and at least one processor in communication with the transceiver which receives input from the pH sensor and wherein said processor is configured to deliver a signal to the reservoir to release a medicament that it contains when

10. The system of claim 9, wherein said device further comprises at least one biopsy arm in communication with the processor.

11. The system of claim 9, wherein said at least one processor is in communication with a valve oriented so as to prevent retrograde flow of stomach contents into the lower esophagus when closed, but to permit passage of material from the lower esophagus into a stomach when open, wherein said processor is configured to deliver a signal to the valve to open or close when predetermined conditions are present.

12. The system of claim 9, further comprising:
a link to a network and/or printer, display, mobile phone and/or other remote device.

13. A method for diagnosing or monitoring a patient for GERD or another esophageal disorder comprising positioning the device of claim 1 in the lower esophagus of the patient, and receiving output from the pH sensor and a camera, when present on the device [, and optionally processing or storing the output.

14. The method of claim 13, wherein the patient having GERD or another esophageal disorder is selected when a pH of the lower esophagus is less than 4 and/or when a camera image indicates inflammation or tissue damaged by acid in the lower esophagus.

15. The method of claim 13, further comprising obtaining a biopsy of esophageal tissue using one or more biopsy arms on the device, and selecting the patient having GERD or another esophageal disorder when a pH of the lower esophagus is less than 4 and/or when a camera image indicates inflammation in the lower esophagus, and/or a camera image indicates eosinophilia of the lower esophagus, and/or when a camera image shows precancerous or cancerous cells in a biopsy.

16. The method of claim 13, wherein the patient has GERD.

17. A method for treating a patient for GERD or another esophageal disorder comprising positioning the device of claim 1 in the lower esophagus of the patient, sending output from the pH sensor or image output from a camera on the device to the transceiver, and when a pH of less than 4 is detected in the esophagus, delivering a signal to the reservoir to release the medicament and/or delivering a signal to a valve on the device to close thus preventing acid reflux from the stomach into the esophagus.

18. The method of claim 17, wherein the patient has GERD.

19. The method of claim 17, wherein the medicament comprises an acid neutralizing material.

20. The method of claim 17, wherein the medicament comprises a foaming agent, H2 blocker, a proton pump inhibitor, or a prokinetic drug.
